(19) United States
(12) Patent Application Publication
(10) Pub. No.: US 2007/0276196 A1
(43) Pub. Date: Nov. 29, 2007

(54) Single acquisition system for electrophysiology and hemodynamic physiological diagnostic monitoring during a clinical invasive procedure

(75) Inventors: Brenda L. Donaldson, Harrison Township, MI (US); Sachin Vadodaria, Fox Point, WI (US)

Correspondence Address: GE MEDICAL SYSTEM;C/O FOLEY & LARDNER LLP, 777 EAST WISCONSIN AVENUE, MILWAUKEE, WI, 53202-5306, US

(73) Assignee: General Electric Company

(21) Appl. No.: 11/434,049

(22) Filed: May 15, 2006

(51) Int. Cl. A61B 5/00

(52) U.S. Cl. 600/300

(57) ABSTRACT

A single acquisition diagnostic monitoring system for monitoring electrophysiology data and hemodynamic physiological data from a subject of interest. The system comprises a first module configured to receive the electrophysiology data via a plurality of first sensors coupled to the subject of interest. The system also comprises a second module configured to receive the hemodynamic physiological data via a plurality of second sensors coupled to the subject of interest. The system also comprises a base unit coupled to each module and configured to supply electrical power to each module, to receive the data from each module, and to synchronize the data from each module together. The system also comprises a processor coupled to the base unit and configured to receive the synchronized electrophysiology and hemodynamic physiological data from the base unit, combine the synchronized data into a single database, and transmit the synchronized processed data for further use.

### FIELD OF THE INVENTION

This invention relates to medical data acquisition and more particularly to a simple acquisition diagnostic monitoring system for monitoring electrophysiology data and hemodynamic physiological data during clinical invasive procedures.

### BACKGROUND OF THE INVENTION

During clinical invasive procedures such as interventional cardiology or radiology procedures there is a need to continuously monitor the physiologic parameters of the patient. Monitoring a patient requires the measurement and analysis of multiple parameters. Some of these parameters are invasive pressure measurements and invasive voltage and time measurements. All physiological diagnostic monitoring systems available today do not provide both these parameters concurrently. A user must exit one application and enter another one or move from one mode to another to be able to make both these measurements on multiple waveforms being recorded and displayed on multiple channels. This change causes extra time delay and extends the procedure.

In most systems the data collected cannot be visualized together at a single location for raw data analysis and diagnosis. This further impedes the workflow and increases the time taken to provide the needed care for the patient. In certain cases the workflow of the procedure is to do a hemodynamic analysis of the patient followed by an electrophysiology (EP) procedure on the same patient. The initial hemodynamic (HEMO) study is done to look for ischemic causes of arrhythmias, holes in the heart between chambers where holes should not exist, tightening or leakage of the heart's valves, or other such procedures, when no ischemic, or structural cause is identified the patient immediately undergoes an EP procedure. Today this takes a longer time as a user switches from one type of an application to another to complete the case. This increase in case time adds risk of complications for the patient. In addition while the application is being switched the patient is monitored either by trained staff or by staff with another monitoring device. If any pertinent changes happen (such as an arrhythmia) to the patient during this application change the clinician does not have an electronic copy of the event to compare against any arrhythmias found during the remainder of the procedure.

Multiple pieces of equipment in a room add to the ambient electrical noise level of a room. Cardiac electrophysiology equipment takes small electrical signals (less than 50 Hz) and amplifies them for the clinician to evaluate. While properly grounded equipment without leakage does not add interference, the more pieces of equipment in a room the more difficult it is to ascertain the source of a background noise. Additionally when a clinician changes from one type of procedure to another using the same computer it is not always obvious to the user that the hardware being accessed has changed.

Currently available systems in an electrophysiology lab require taking the patient off of the transportation monitor and monitoring them via the EP amplifier. If an arrhythmia occurs during this transition, limited documentation will be available to the clinician to assess the arrhythmia. When a procedure is changed from a hemodynamic or electrophysiological procedure to the other type of procedure the equipment must be recalibrated and rezeroed. While recalibration is in progress other functions of the system are not available preventing the user from proceeding with the procedure and delaying treatment. Since no physical change in connections is required vital time may be lost while the clinician ascertains the source of the loss of data and goes through the calibration process.

Thus there is a need for a single acquisition system for both electrophysiology and hemodynamic physiological diagnostic monitoring during a clinical invasive procedure. There is also a need for a tool for monitoring electtophysiology data and hemodynamic physiological data of a subject of interest in a single database using a processor.

### SUMMARY OF THE INVENTION

One embodiment of the invention relates to a single acquisition diagnostic monitoring system for monitoring electrophysiology data and hemodynamic physiological data from a subject of interest. The system includes a first module configured to receive the electrophysiology data via a plurality of first sensors coupled to the subject of interest. The system also includes a second module configured to receive the hemodynamic physiological data via a plurality of second sensors coupled to the subject of interest. The system also includes a base unit coupled to each module and configured to supply electrical power to each module, to receive the data from each module, and to synchronize the data from each module together. The system also includes a processor coupled to the base unit and configured to receive the synchronized electrophysiology and hemodynamic physiological data from the base unit, combine the synchronized data into a single database, and transmit the synchronized processed data for further use.

Another embodiment of the invention relates to a method of monitoring electrophysiology data and hemodynamic physiological data from a subject of interest using a first module configured to receive the electrophysiology data and a second module configured to receive the hemodynamic physiological data, with each module coupled to a base unit and a processor. The method includes receiving the electrophysiology data, receiving the hemodynamic physiological data, synchronizing the two data sets and combining the synchronized data sets into a single database, wherein the single database is available for further use.

Another embodiment of the invention relates to a tool for monitoring electrophysiology data and hemodynamic physiological data of a subject of interest using a processor. The tool includes a means for receiving a electrophysiology data set from the subject of interest. The tool also includes a means for receiving a hemodynamic physiological data set from the subject of interest. The tool also includes a means for synchronizing the two data sets and a means for combining the synchronized data sets into a single database in the processor, wherein the single database is available for further use.

### BRIEF DESCRIPTION OF THE DRAWINGS

FIG. 1 is a schematic diagram of an exemplary embodiment of a single acquisition diagnostic monitoring system for monitoring and processing electrophysiology data and hemodynamic physiology data from a subject of interest.

FIG. 2 is a schematic diagram of an exemplary embodiment of a tool for monitoring and processing the electrophysiology data and hemodynamic physiology data from a subject of interest.

### DETAILED DESCRIPTION OF THE EXEMPLARY EMBODIMENTS

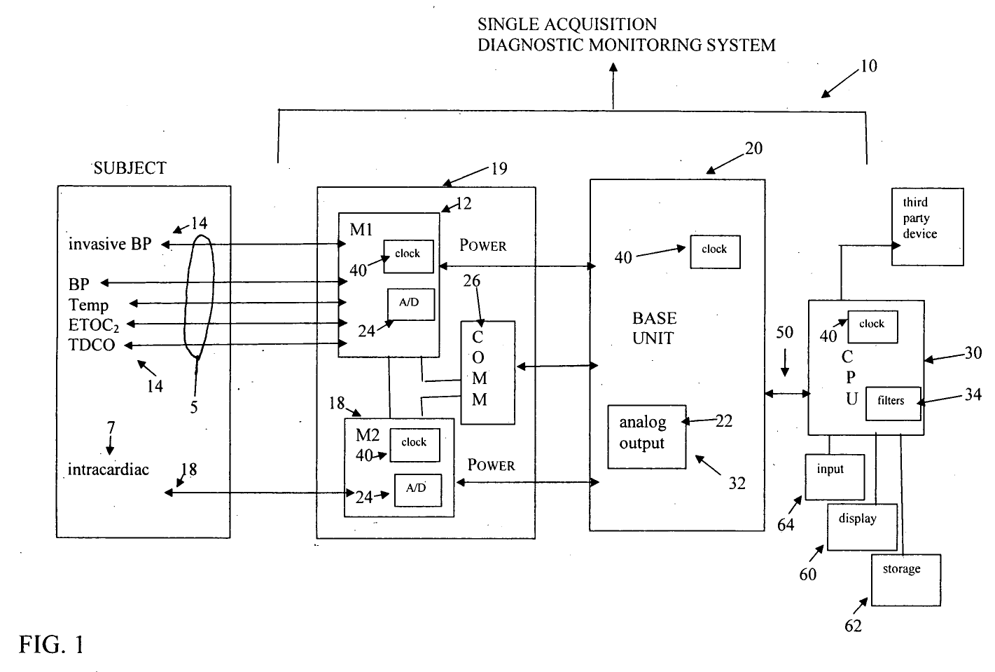

Referring to the figures, FIG. 1 illustrates an exemplary embodiment of a single digital acquisition system 10 which uses one set of patient connections to collect both hemodynamic 7 (pressure and vitals) and electrophysiologic 5 (electrical) data sets simultaneously from the subject of interest at once and filters and communicates digitally to a computer 30 that has a software tool 70 to process this for display, analysis, storage, and further use.

The system 10 allows for the smooth transition from a hemodynamic case to an electrophysiology ablation procedure in the same setting.

In one exemplary embodiment, subject of interest may be a human. In other exemplary embodiments, subject of interest may be another anatomical structure such as a dog, cat, horse, or primate. In another exemplary embodiment, the system 10 is included in a patient care facility for example a hospital or hospital room, while in still other exemplary embodiment, the facility may be any facility suitable for performing medical procedures, for example imaging, invasive procedures, or diagnostic procedures, etc, on the subject of interest.

The single acquisition diagnostic monitoring system 10 for monitoring electrophysiology data 5 and hemodynamic physiology data 7 from a subject of interest includes a first module 12 configured to receive the electrophysiology data 5 via a plurality of first sensors 14 coupled to the subject of interest. A second module 16 is configured to receive the hemodynamic physiological data via a plurality of second sensors 18 coupled to the subject of interest. The data sets 5 and 7 are received by a base unit 20 coupled to each of the modules 12, 16 and configured to supply electrical power to each module and to synchronize the data 5, 7 from each module together. A processor 30 is coupled to the base unit 20 and configured to receive the synchronized electrophysiology and hemodynamic physiology data from the base unit 20, combine the synchronized data into a single database, and transmit the synchronized processed data for further use.

The first module 12 obtains data from the subject of interest such as ECG signals, invasive blood pressures, non-invasive blood pressure, temperature monitoring, end title carbon dioxide and thermodilution cardiac output (TDCO). The module may sample, as determined by the user, at a 16,000 sampling rate and the data may be down sampled to 500 k or 240 k depending on the stream selected by the processor 30 (as an example, hemodynamics vs. electrophysiological procedures). Data is used in the module to analyze the ECG signals looking for pacing spikes, when pacing spikes are detected, the spikes are not counted as a heartbeat. The data is collected via a plurality of sensors that are configured to obtain the desired data.

The second module 16 obtains intracardiac signals via a plurality of second sensors 18 such as catheters at a preselected sampling rate. Typically no filtering or amplification of the intracardiac signals are done at the module or at the base unit 20.

Additional modules that may be coupled to the other modules in the base unit 20.

The base unit 20 is coupled to each of the modules and provides electrical power to the modules. The power can be supplied to each of the modules directly or it can be provided to one module with a number of slave modules coupled to the primary module.

Each module 12, 8 and the base unit 20 together with the processor 30, includes a clock 40. A standard communications protocol is used to synchronize the clocks 40 of the modules, the base unit and the CPU. The data received by the modules 12, 18, is time stamped by each module. Each module 12, 14 performs an analog to digital conversion in an analog/digital circuit 24 and transmits the converted data to the base unit 20. The base unit 20 upon receipt of the data utilizes the time stamps to synchronize the data and converts the data into standard TPCP/IP packets. The packets are then forwarded as Ethernet packets to the processor 30 and to an analog output circuit 22 which is coupled to the base unit 20. A wireless network, for example a Bluetooth system, may also be used.

The processor 30 which can be any kind of central processing unit such as a laptop computer or a desktop computer, or a server-type computer, receive the synchronized electrophysiology data 5 and the hemodynamic physiology data 7 from the base unit 20. The processor combines the synchronized data into a single database for further processing.

The analog output circuit 22 applies filters 34 to the data. The filters 34 which can be a high pass filter, a low pass filter, or a combination of high and low pass filters are selected from a group stored and generated on the processor 30. The user of the system 10 can select those filters on the processor 30 and are transmitted to the analog output circuit 22. The analog output circuit 22 will apply a; predetermined filter 32 received from the processor 30 to the process data. It should be understood that the filters 34 can be changed by well known programming techniques on the processor 30 from time to time.

The processor 30 also includes an input device 64 which can be, for example, a keypad, a keyboard, a joystick, a roller ball, a touch pen, or a voice recognition system. Also coupled to the processor 30 is a display unit 60. The display unit 60 can include three separate monitors that are configured to concurrently display real time wave form data from the subject of interest, stored wave form data of the subject of interest and live or saved physiological images of the subject of interest.

The collected data and analyzed data and annotated data can be stored in a storage unit 62 coupled to the processor 30 and can be, for example, a hard drive, another computer, a tape or disk data storage media.

There is also provided a method of monitoring electrophysiology and hemodynamic physiological data 5, 7 on the subject of interest using a first module 12 configured to receive the electrophysiological data 5 and a second module 16 configured to receive the hemodynamic physiological data 7 with each module 12, 16 coupled to a base unit 20 and a processor 30. The method includes receiving the electrophysiological data, receiving the hemodynamic physiological data 7 and synchronizing the two data sets. Combining the synchronized data sets into a single database, wherein the single database is available for further use.

The method may also include the step of generating a filter 32 in the processor 30 and applying the synchronized data sets to the filter 32. The filter 32 is selected from a group 34 consisting of a low pass filter, a high pass filter and a combination of low and high pass filters. The filters can be programmed into the processor 30 as determined by a user or it may be selected from a table stored on the processor 30.

In a system 10 for monitoring electrophysiology and hemodynamic physiological data 5, 7, each of the modules 12 and 16, the base unit 20 and the processor 30 include a clock 40. The method includes a step of synchronizing the clock 40 in each of the modules 12, 16, the base unit 20 and the processor 30. The synchronization of the data provides a means for date stamping the data received from the modules to the base unit and to the processor 30.

The synchronized data sets are transmitted by the computer to a third party device or to a display unit 60, or a storage unit 62. It is also possible for the data to be provided in an analog output device 22 such as a strip chart.

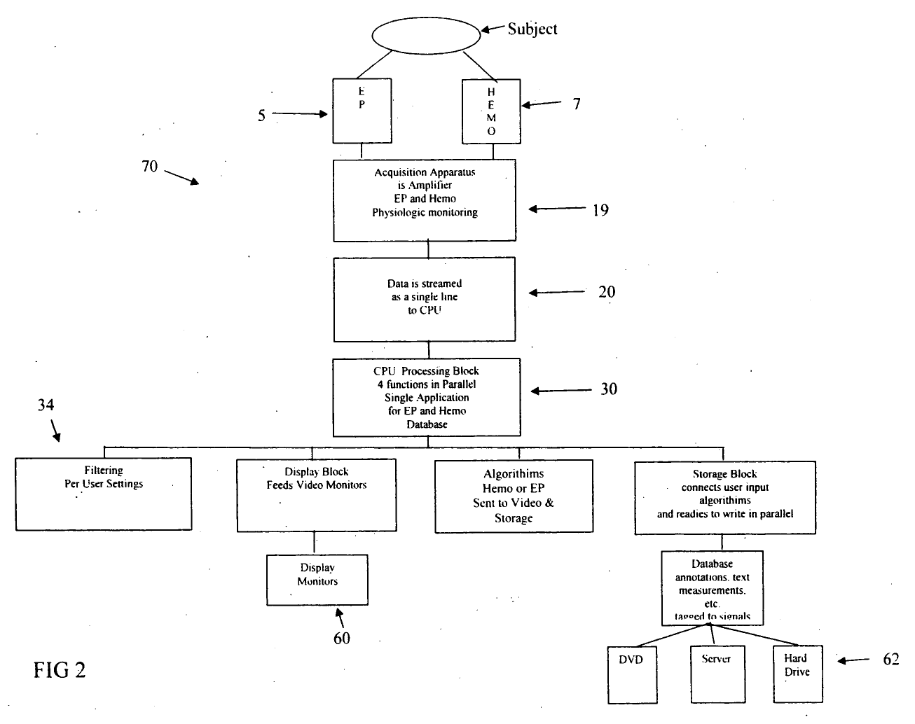

The system 10 includes a tool 70 for monitoring electrophysiology and hemodynamic physiological data 5, 7 for the subject of interest using a processor 30. The tool 70 includes a means for receiving an electrophysiology data set from the subject of interest. It also includes a means for receiving hemodynamic physiological data set from the subject of interest and the means for synchronizing the two data sets 5,7. The tool 70 also includes a means for combining the synchronized data sets into a single database in the processor wherein the single database is variable for further use. In the processor 30 several functions are performed in parallel on the single application of EP and HEMO data sets. As illustrated in FIG. 2, the filtering of the data as selected by the user is performed. A display of selected data is sent to video monitors 6 which are coupled to the processor 30. Analogous algorithms are applied to the HEMO or EP data sets as selected by the user and sent to the display monitor 60 or to a storage unit 62. The storage units are coupled to the processor 30. The storage units contain the raw and analyzed and imitated data in a single database. The display unit 60 can be configured to concurrently display real time waveform data from the subject of interest, stored waveform data on the subject of interest and live or saved physiological images of the subject of interest. The display unit 60 may be a single screen, for example a large plasma or liquid crystal display unit, or multiple screens. In each case, the display unit 60 must have a rapid refresh rate to provide a suitable image and text display for the user.

In one exemplary embodiment, the system 10 will acquire, display and analyze multi-parameters simultaneously concurrently on a single user interface to the user. The system 10 will provide both hemodynamic (hemo) measurement and analysis capability such as multi-invasive blood pressure measurements, and vitals data collection, such as Non-Invasive Blood Pressure, Oxygen Saturation, Heart Rate, etc. along with electrophysiology (ep) measurement and analysis such as voltage data measurements and therapy data collection such as radio frequency ablation parameters. In addition, the system 10 may be configured to receive images from other coupled devices, for example x-ray, ultrasound, archived images and 3-D cardiac mapping systems. The images from such systems can be stored in the same database coupled to the processor 30. If such images are acquired during the procedure in which the system 10 is being used, then the physiologic signals from the subject of interest will be tagged to these images so that the user may select to view a selected image and see the waveform that occurred at the same time period.

The waveforms of the acquired data will be concurrently displayed on a single screen or window with up to 4 invasive pressure measurements and 128 intracardiac electrophysiology waveforms on discrete display channels. The display will include 12 lead surface electrocardiograms (ECG) data, non-invasive blood pressures and other hemodynamic waveforms such as pulse oximetry for determining the vital status of the patient. The single application will be able to store these simultaneously from the beginning of the case to the end of the case with a time stamp at each point of acquisition. The saved data will be displayed on a second screen in a separate window concurrently. The system will be able to measure and analyze simultaneously the data sets on a separate window or screen that displays saved or stored datasets. These measurements can include basic blood pressure measurements, blood flow across heart valves (valve-area) measurements, blood flow from one chamber to another through a hole (shunt calculations), thermodilution cardiac outputs, intracardiac and surface electrocardiograms (ECG) voltage measurements and radio frequency ablation parameters such as voltage, power, temperature and impedance. The acquisition of these waveforms may be in multiple frequencies from 250 Hz to 10 KHz as defined by the user synchronously displayed on one screen in live mode or saved mode. The user can measure and manipulate the signals using controls available on the system such as amplification, annotation, side by side comparison, maps, etc. using several types of input devices 64. The system has well known software algorithms that automatically calculates pressure and electrical data sets as well as more complex parameters such as slope of the pressure curve (dp/dt), etc. The user will have a single window (the log) where notes, measurements, medications, etc are recorder across the procedure. This ability then assists the user in the clinical decision making process for the patient's health. These stored (on the hard drive, a removable medium such as DVD or to a server) signals can then be then printed electronically on an electronic file or physically printed on a hard copy using a printer.

By allowing access to both hemodynamic and electrophysiology analysis tools simultaneously on the pressure and electrical waveforms acquired simultaneously the user would be able to seamlessly transition or integrate between multiple clinical procedures such as invasive cardiac catheterization and invasive electrophysiology procedures thereby reducing time to complete the procedures and facilitating currently awkward or difficult work flows such as the previously discussed repair of holes in the heart in combination with an ablation procedure. The user would also be able to better document the complete case and have a single congruent record of the patient's status at the end of the case which would encompass both types of procedures.

The system 10 acquires directly from the subject of interest using sensors 14, 18, for example, surface patches located on the chest and the body as well as intra body catheters (hollow tubes for hemodynamics and closed lumen for electrophysiologic procedures) inserted through various access points (femoral vein, femoral artery (for hemo) jugular vein, etc.) which have sensors arrays mounted on them to record pressure and voltage data and then translate that to waveforms that are displayed on the display units 60 of the system 10 for the user to visualize. The user then uses the system 10 to analyze data sets recorded to ascertain the patient's health and make clinical decisions. Finally the systems generates a comprehensive report of the procedure that can be printed, stored digitally and sent to the hospital information system for archival.

The data obtained during the procedure may include waveforms of pressures, calculations of such things as pressure measurements, valve calculations, shunt calculations, snapshots of x-ray images, snapshots of ultrasound images or loops of ultrasound images, electrophysiologic measurements such as A-H and H-V (the time it takes for a signal to go from the sinus node in the atrium to the ventricle) this data is initially stored on the hard drive with in a buffer, as CPU processing power becomes available the data is stored on a removable medium (such as DVD) and may optionally also be written to a server.

For purposes of this disclosure, the term “coupled” means the joining of two components (electrical or mechanical) directly or indirectly to one another. Such joining may be stationary in nature or movable in nature. Such joining may be achieved with the two components (electrical or mechanical) and any additional intermediate members being integrally defined as a single unitary body with one another or with the two components or the two components and any additional member being attached to one another. Such joining may be permanent in nature or alternatively may be removable or releasable in nature

The present disclosure has been described with reference to example embodiments, however workers skilled in the art will recognize that changes may be made in form and detail without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example is manifestly intended to be as broad as possible. For example, unless specifically otherwise noted a single particular element may also encompass a plurality of such particular elements.

It is also important to note that the construction and arrangement of the elements of the system as shown in the preferred and other exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes and omissions may be made in the design, operating conditions and arrangement of the preferred and other exemplary embodiments without departing from the spirit of the present subject matter.

What is claimed is:

1. A single acquisition diagnostic monitoring system for monitoring electrophysiology data and hemodynamic physiological data from a subject of interest, the system comprising:
a first module configured to receive the electrophysiology data via a plurality of first sensors coupled to the subject of interest;
a second module configured to receive the hemodynamic physiological data via a plurality of second sensors coupled to the subject of interest;
a base unit coupled to each module and configured to supply electrical power to each module, to receive the data from each module, and to synchronize the data from each module together; and
a processor coupled to the base unit and configured to receive the synchronized electrophysiology and hemodynamic physiological data from the base unit, combine the synchronized data into a single database, and transmit the synchronized processed data for further use.

2. The system of claim 1, including an analog-out circuit coupled to the base unit and the processor, with the circuit configured to apply a predetermined filter received from the processor to the processed data.

3. The system of claim 2, wherein the processor is configured to generate the predetermined filter from a group consisting of low pass, high pass, and a combination of low and high pass filters available to the processor.

4. The system of claim 1, including a clock in each of the modules, base unit and processor, with all the clocks synchronized with a communication protocol.

5. The system of claim 1, wherein the processor and base unit are coupled together by one of a hardwire cable and a wireless network.

6. The system of claim 1, wherein each of the modules includes an analog-to-digital converter to process the data each receives from the sensors before transmitting the data to the base unit.

7. The system of claim 1, including a display unit coupled to the processor and configured to concurrently display real time waveform data from the subject of interest, stored waveform data of the subject of interest, and live or saved physiological images of the subject of interest.

8. The system of claim 7, wherein the display unit includes three separate monitors.

9. A method of monitoring electrophysiology data and hemodynamic physiological data from a subject of interest using a first module configured to receive the electrophysiology data and a second module configured to receive the hemodynamic physiological data, with each module coupled to a base unit and a processor, the method comprising:
receiving the electrophysiology data;
receiving the hemodynamic physiological data;
synchronizing the two data sets; and
combining the synchronized data sets into a single database, wherein the single database is available for further use.

10. The method of claim 9, including the steps of generating a filter in the processor and applying the synchronized data sets to the filter.

11. The method of claim 10, wherein the filter is selected from a group consisting of a low pass filter, a high pass filter, and a combination of low and high pass filters.

12. The method of claim 9, including the step of synchronizing a clock in each of the modules, the base unit and the processor.

13. The method of claim 9, including the step of transmitting the synchronized data sets for further use.

14. The method of claim 9, including the step of displaying the synchronized data sets on a display unit coupled to the processor, with the display unit configured to concurrently display real time waveform data from the subject of interest, stored waveform data of the subject of interest, and live or saved physiological images of the subject of interest.

15. The method of claim 14, wherein the display unit includes three separate monitors.

16. A tool for monitoring electrophysiology data and hemodynamic physiological data of a subject of interest using a processor, the tool comprising:
means for receiving a electrophysiology data set from the subject of interest;
means for receiving a hemodynamic physiological data set from the subject of interest;
means for synchronizing the two data sets; and
means for combining the synchronized data sets into a single database in the processor, wherein the single database is available for further use.

17. The tool of claim 16, including a means for generating a filter in the processor and a means for applying the synchronized data sets to the filter.

18. The tool of claim 17, wherein the filter is selected from a group consisting of a low pass filter, a high pass filter, and a combination of low and high pass filters.

19. The tool of claim 16, including a display unit coupled to the processor, with the display unit configured to concurrently display real time waveform data from the subject of interest, stored waveform data of the subject of interest, and live or saved physiological images of the subject of interest.

20. The tool of claim 19, wherein the display unit includes three separate monitors.
